(19) United States
(12) Plant Patent Application Publication
(10) Pub. No.: US 2003/0126659 P1
(43) Pub. Date: Jul. 3, 2003

(54) Curcuma Plant Named 'Laddawan'

(76) Inventor: Pichai Manichote, Bangkok (TH)

Correspondence Address: CHRISTIE, PARKER & HALE, LLP, 350 WEST COLORADO BOULEVARD, SUITE 500, PASADENA, CA, 91105, US

(21) Appl. No.: 10/037,764

(22) Filed: Jan. 2, 2002

(51) Int. Cl. A01H 5/00

(52) U.S. Cl. PLT/263

(57) ABSTRACT

A new and distinct cultivar of Patumma named ‘LADDAWAN’ characterized by green bracts, purplish pink coma bracts on a cylindical inflorescence and florets with yellow labellum.

### BACKGROUND OF THE INVENTION

The present invention is directed to a new and distinct cultivar of Patumma, botanically know as Curcuma hybrid, and having the cultivar name ‘LADDAWAN’.

The new cultivar was a seedling among a progeny of Curcuma alismatifolia×Curcuma cf. cordata, selected by the inventor.

Asexual propagation of the new cultivar in Nakhon Pathom, Thailand by division and micro-propagation has demonstrated the uniformity and the stability throughout successive generations of this new Patumma.

### SUMMARY OF THE INVENTION

The following traits have been repeatedly observed in the new cultivar grown under light shade condition in Nakhon Pathom, Thailand which closely approximate those commonly used in commercial horticultural practice.

The features that in combination are believed to distinguish ‘LADDAWAN’ from other known cultivars of Patumma include: long peduncle; purplish pink coma bracts, obovate bracts; oblong ovate leaves.

Plants of the new Patumma can be compared to plants of the Curcuma alismatifolia ‘CHIANG MAI’. Plants of the new Patumma differ from plants of the cultivar ‘CHIANG MAI’ in the following characteristics:

1. The labellum of the new Patumma floret is yellow, instead of blue as found in ‘CHIANG MAI’.

2. The coma bract of the new Patumma is elliptic and spacing apart from each other, instead of obovate and closing together as found in ‘CHIANG MAI’.

3. The leaf of the new Patumma is oblong ovate, instead of elliptic as found in ‘CHIANG MAI’.

4. The lateral vein of the new Patumma is pronounced, instead of diffused as found in ‘CHIANG MAI’.

### BRIEF DESCRIPTION OF ILLUSTRATIONS

The accompanying color photographs illustrate the overall appearance of the new cultivar as true in color as possible in color illustration of this type.

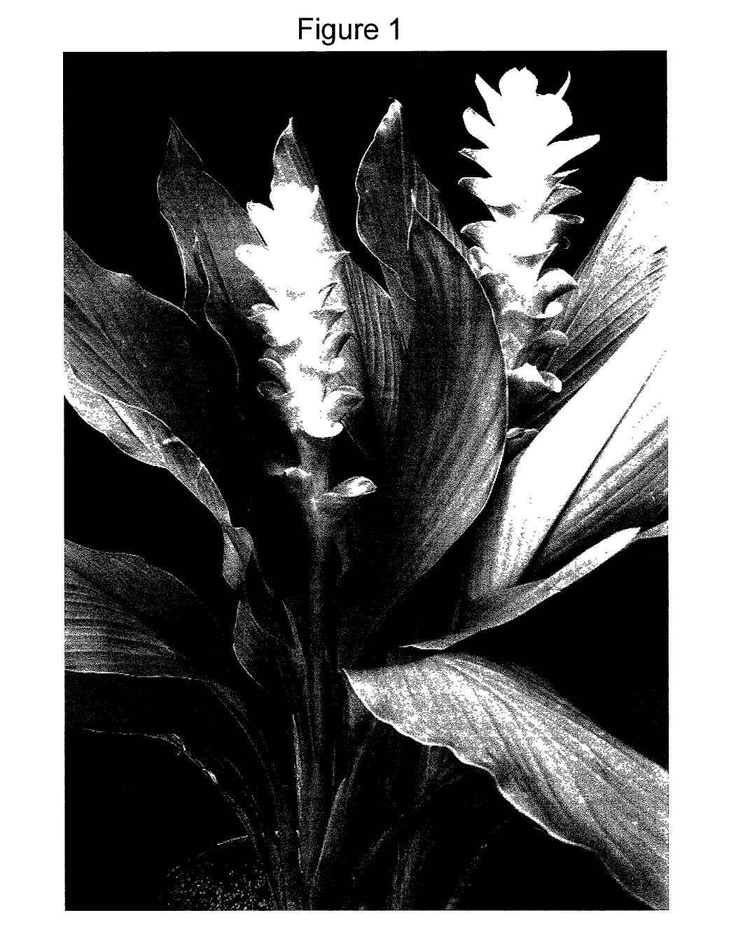

FIG. 1 is a side view of a full-grown plant of ‘LADDAWAN,’ illustrating both leaves, inflorescence and florets.

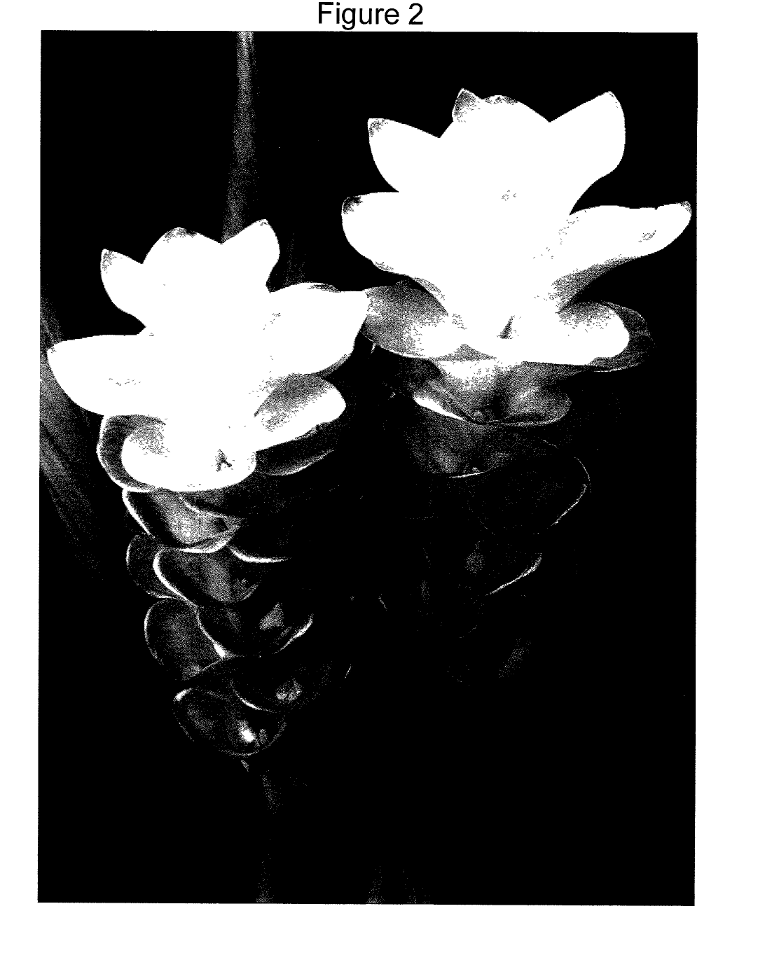

FIG. 2 is an inflorescence and florets of Curcuma hybrids ‘LADDAWAN’.

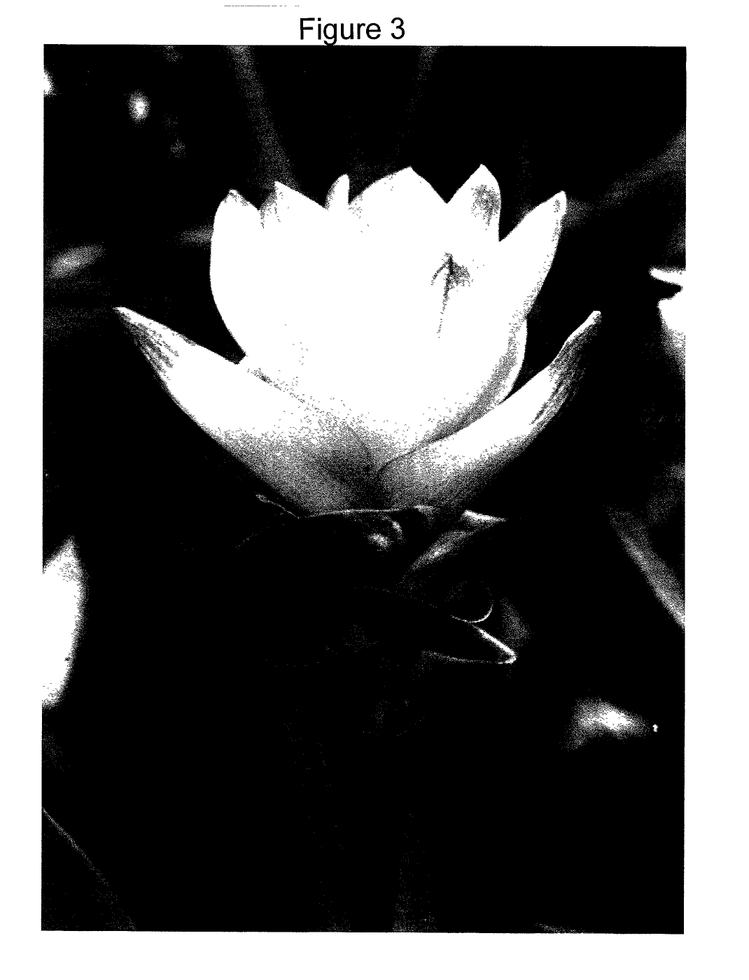

FIG. 3 is an inflorescence of Curcuma alismatifolia ‘CHIANG MAI’

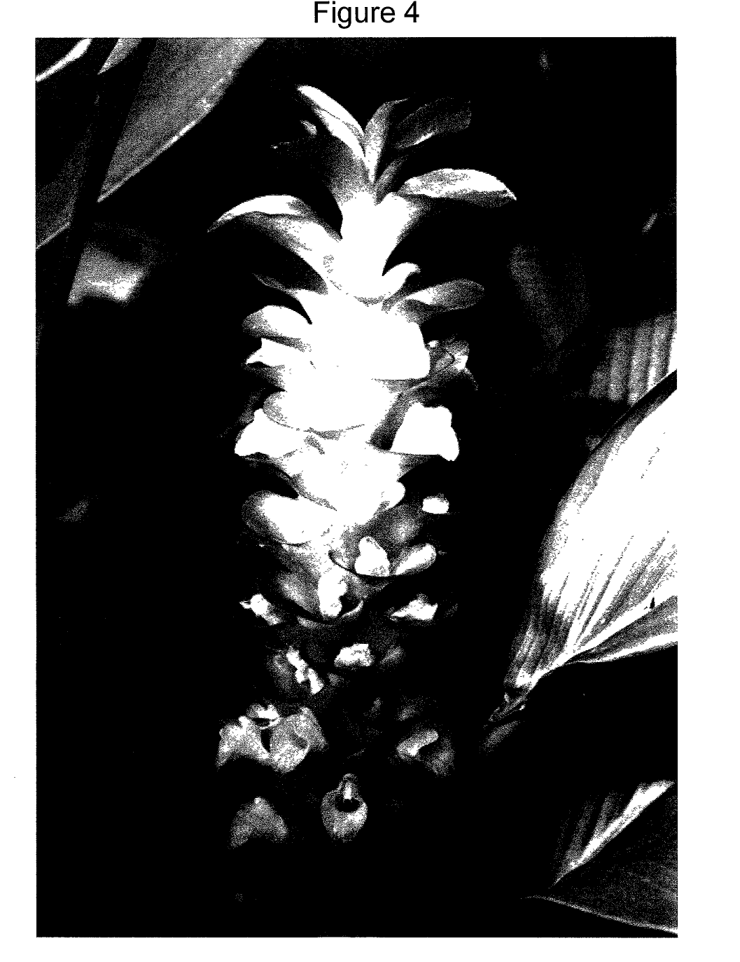

FIG. 4 is an inflorescence of Curcuma cf. cordata.

### DESCRIPTION OF THE NEW VARIETY

The following observations were made of 5-month old plants of the new variety grown under light shade conditions, in Nakhon Pathom, Thailand during the months of April through September. Phenotypic expression may vary with environmental, cultural and climatic conditions, as well as differences in conditions of light and soil.

Throughout this specification, color references and/or values are based upon The Royal Horticultural Society Colour Chart and terminology used in the color descriptions herein refers to plate numbers in this color chart except where common terms of color definition are employed.

Parentage:

Female parent.—Curcuma alismatifolia.

Male parent.—Curcuma cf. cordata.

Propagation: By division and micro-propagation.

Plant:

Shape.—Pseudostem upright and spreading.

Growth habit.—Resulting from branching of rhizome, the new pseudostem emerges close to the old pseudostem.

Size.—Height from soil surface to top of leaf canopy about 40 to 50 cm; height from soil surface to the highest leaf axil about 13 to 23 cm.

Rhizome description.—Corm-like; diameter of 2 to 3 cm, height 2.2 to 4.5 cm; flesh near 4C.

Vigor.—Vigorous, rapid growth rate (multiplication rate of 7 to 16 folds per season).

Foliage:

Leaf.—Shape: Oblong ovate with an attenuate apex, an obtuse base and an entire margin Size: Length about 32 to 38 cm; width about 11.8 to 15.6 cm Surface: Smooth with pronounced veins Aspect: Distichous alternate array of leaf petiole on pseudostem. Erect initially, eventually arch outward.

Petiole.—Length about 2.4 to 4 cm.

Inflorescence: From apical bud of pseudostem, green peduncle of about 13.5 to 22 cm long; rachis about 17.5 to 22.7 cm long and about 5 to 7.7 cm diameter; 12 to 17 coma bracts, coma bract color near 75A, obovate; 18 to 27 bracts, bract color near 144C, obovate; waxy coma bracts and bracts

Floret.—About 3.7 to 4 cm long; calyx tube about 1 to 1.2 cm long with 3 lobes, corolla tube about 1.9 to 2.2 cm long with 3 unequal lobes, smaller side lobe; side staminode near 4D, about 1.4 to 1.7 cm long, 0.9 to 1 cm wide; labellum near 4D, about 1.7 to 1.9 cm wide, gills near 3A, crest tip; anther with distal anther crest.

Plant of the Patumma cultivar ‘LADDAWAN’ is upright and clumping. The inflorescence, from pseudostem apex, is cylindical with open top and has green bracts and purplish pink coma bracts. Floret has a yellow labellum with dark yellow gills.

I claim:

1. A new and distinct plant of Patumma named ‘LADDAWAN’ substantially as illustrated and described.
